(19) United States
(12) Patent Application Publication
(10) Pub. No.: US 2015/0157414 A1
(43) Pub. Date: Jun. 11, 2015

(54) PERSONAL HEALTHCARE DEVICE

(71) Applicant: Greg Van Curen, Fremont, IN (US)

(72) Inventor: Greg Van Curen, Fremont, IN (US)

(21) Appl. No.: 14/101,984

(22) Filed: Dec. 10, 2013

(51) Int. Cl. A61B 19/00

(52) U.S. Cl. CPC A61B 19/36

(57) ABSTRACT

A personal healthcare device includes a base member mountable to a wall and at least one insert. Each insert has a size and shape corresponding to a size and shape of a corresponding recess in the base member. Each insert further includes at least one protrusion extending from the insert in a direction away from the base member. Additionally, each protrusion is configured for selectively making contact with and applying pressure to a surface of a user.

### BACKGROUND OF THE INVENTION

1. Field of the Invention

The present invention relates to a personal healthcare device.

2. Description of the Related Art

There are a number of devices on the market for relieving musculoskeletal discomfort and/or itching related to dry skin, insect bites, allergic reaction or other skin-related afflictions. Depending on the location of the afflicted area, an individual suffering from such irritations may be able to address it without the aid of a third party, for example in areas which are on the front of the body or within easy reach, such as on the arms, legs, chest or feet. Unfortunately, not all surface areas of the body are readily accessible by the individual without the aid of either a third party or a device which operates as an extension of the arm of the individual. The most readily identifiable such area which is particularly difficult to reach is on one's back.

A number of devices have been developed in the art to make it easier for a user to address discomfort, whether it be itching or related to a neuromuscular condition. For example, a number of hand-held devices have been developed for the application of pressure to a predetermined area of the body. An example of such a hand-held device is a percussion massager which applies a steady progression of pulses of pressure against the desired area. The use of such a hand-held device is, however, limited by the user's dexterity, unless assisted by a third party. Additionally, elongate back scratchers and massagers are known in the art. Typically, these are in the form of an elongate handle with a curved prong or plurality of prongs extending therefrom. The user then extends the apparatus over his or her shoulder and applies pressure while trying to position the device over the desired area. The problem with this type of device is that the amount of pressure which can be applied to the desired area is limited by the user's flexibility and upper body strength, as well as his or her coordination in positioning the device accurately without visual aid.

What is needed in the art is a personal healthcare device which allows the user to selectively apply pressure to the affected area without the associated problems of the known art.

### SUMMARY OF THE INVENTION

The present invention provides a personal healthcare device including a base member, mountable to a wall and having at least one recess and at least one insert sized and shaped to correspond with the at least one recess. The at least one insert includes at least one protrusion extending from the insert and in a direction away from the base member. The at least one protrusion is configured for selectively making contact with and applying pressure to a surface of the user.

The present invention also provides a method of producing a personal healthcare device. According to the method of the present invention, a base member which is mountable to a wall and which includes at least one recess is provided. At least one insert having a size and shape corresponding with a size and shape of the at least one recess in the base member is also provided. The at least one insert includes at least one protrusion extending from the insert in a direction away from the base member. The at least one protrusion is configured for selectively making contact with and applying pressure to a surface of the user. The at least one insert is securely positioned within the at least one corresponding recess.

An advantage of the present invention is a user can selectively apply pressure to an otherwise unreachable area of the body without the assistance of another person.

Another advantage is that the selective pressure can be applied to the desired area without requiring direction to a third party, thus saving time and likely discomfort.

Yet another advantage is that a single device can be utilized to manipulate the desired surface of the user to quickly and efficiently relieve uncomfortable physical irritation, such as itches and musculoskeletal aches and pains. Additionally, since the user controls the amount of pressure applied, additional irritation from excessive pressure can be avoided. Upon the achieving relief from the particular itch or musculoskeletal irritation, the user can immediately cease use of the device if desired and avoid the infliction of additional discomfort resulting from excessive pressure or any indication of increased discomfort resulting from misalignment of the device with the affected surface area of the user.

### BRIEF DESCRIPTION OF THE DRAWINGS

The above-mentioned and other features and advantages of this invention, and the manner of attaining them, will become more apparent and the invention will be better understood by reference to the following descriptions of embodiments of the invention taken in conjunction with the accompanying drawings, wherein:

FIG. 1 is a schematic illustration of the personal healthcare device according to an embodiment of the present invention;

FIG. 2 is an exploded view of the personal healthcare device of FIG. 1;

FIG. 3 is a side view of an embodiment of an insert for the personal healthcare device of FIG. 1;

FIG. 4 is a side view of another embodiment of an insert for the personal healthcare device of FIG. 1;

FIG. 5 is a top view of the personal healthcare according to the present invention with a third embodiment of the insert;

Corresponding reference characters indicate corresponding parts throughout the several views. The exemplifications set out herein illustrate embodiments of the invention and such exemplifications are not to be construed as limiting the scope of the invention in any manner.

### DETAILED DESCRIPTION OF THE INVENTION

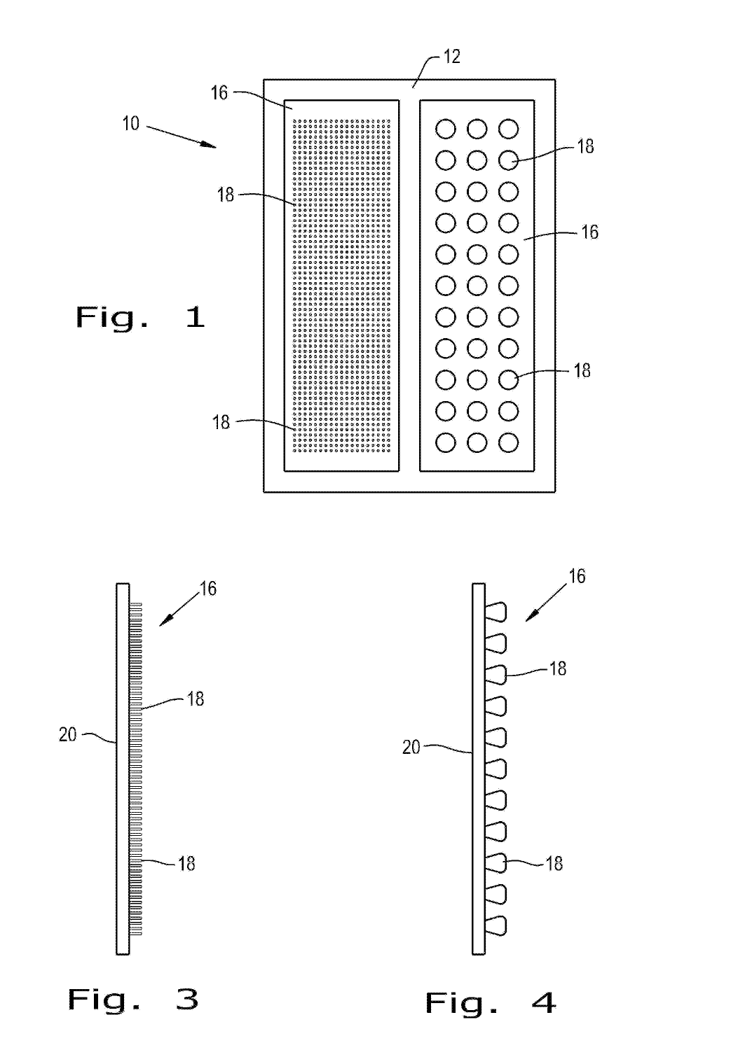

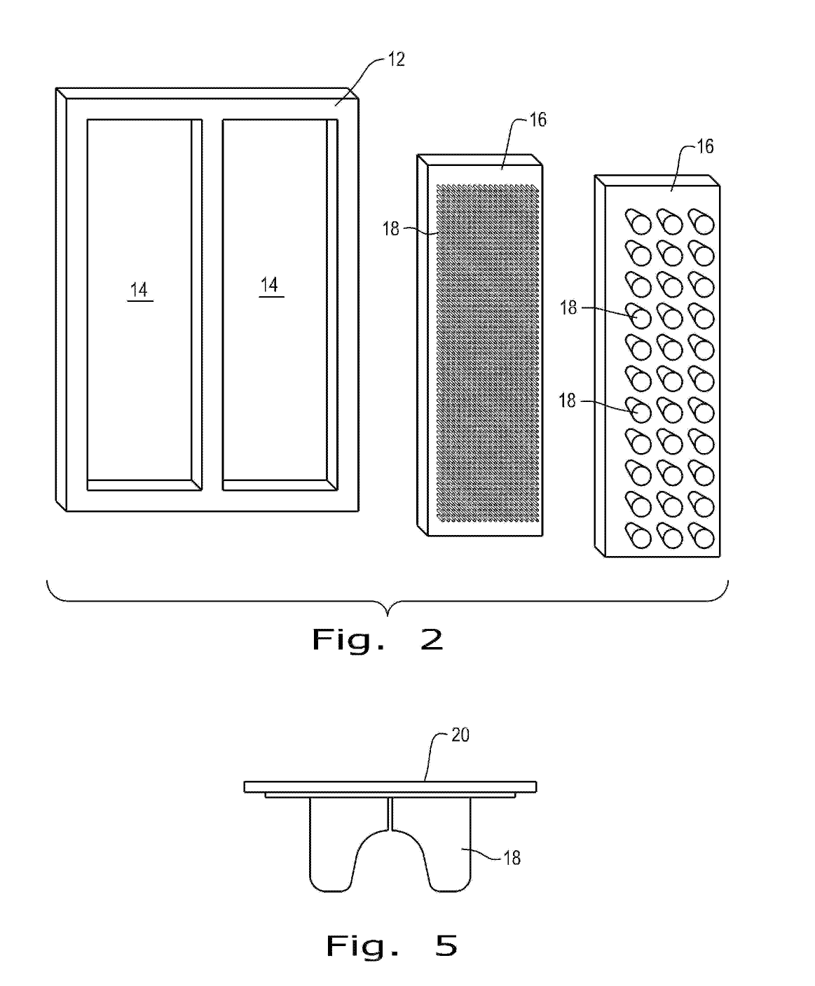

Referring now to the drawings, and more particularly to FIGS. 1 and 2, there is shown a schematic illustration and an exploded view of personal healthcare device 10 of the present invention, which generally includes a base member 12 and one or more inserts 16.

Base member 12 is configured to be securely mounted to a wall, for example, using at least one known fastener. Advantageously, this allows a user to apply increased pressure with little or no movement of personal healthcare device 10. Base member 12 is substantially rigid to avoid bending of device 10 away from the user upon the application of force against one or more protrusions 18 extending from inserts 16 of device 10. Base member 12 may, for example, be formed of wood, metal, plastic or ceramic. Base member 12 includes one ore more recesses 14 which may be in any shape, for example, rectangular, square, kidney-shaped, or round, such as circular or oval-shaped. The one or more recesses 14 may have a length equal to their width. Alternatively, recesses 14 may have a greater width than length or vice versa. Base member 12 may, for example include more than one recess 14, for example two (as shown), three or four recesses 14. If there is more than one recess 14, they may be arranged parallel to each other, transverse to each other, in a regular pattern or in a random pattern.

Inserts 16 have a size and shape of a corresponding recess 14 into which it may be securely positioned. Each insert 16 includes one or more protrusions 18 extending therefrom in a direction away from base member 12. Protrusion 18 extending from insert 16 are configured for selectively making contact with and applying pressure to the surface of a user. Inserts 16 each include a substantially planar portion 20 which is configured to abut an internal surface (not shown) of recess 14. Inserts 16 may be formed from any of a number of materials, including wood, metal, plastic, vulcanized rubber or various polymers. Inserts 16 are sized and shaped to correspond with a corresponding recess 14 in base member 12 and are configured to be securely positioned within corresponding recesses 14 of base member 12. Inserts 16 may be secured within recesses 14 by a number of fastening mechanisms, for example, a snap fit, compression fit, or magnetic fixation. Any known fastener can be utilized to secure inserts 16 into recesses 14, for example, a cam lock, screws, bolts, pins or clips.

Protrusions 18 of inserts 16 extend in a direction away from base member 12 when inserted into recess 14. Recesses 14 may each be sized and shaped to receive a plurality of inserts 16 having differently shaped protrusions 18. For example, according to the present invention, two inserts 16 having differently shaped protrusions 18 may be provided which are interchangeable within a single recess 14 within base member 12.

According to the present invention, protrusions 18 may be configured differently, dependent upon their intended use. A number of exemplary embodiments of inserts 16 including protrusions 18 are illustrated in FIGS. 3-5. According to the embodiments illustrated in FIGS. 3 and 4, inserts 16 are provided which include a plurality of protrusions 18 extending away from base member 12. Protrusions 18 are illustrated in FIG. 3 as being in the form of a plurality of semi-rigid bristles 18 for exfoliating skin from a predetermined surface of the user. In this form, the personal healthcare device 10 may also be effectively utilized to relieve itching. The semi-rigid bristles 18 may be natural bristles, for example, boar bristles or horse hair bristles or they may be man-made from, for example, nylon, stainless steel, plastic, metal or from a plurality of polymer materials.

According to a further embodiment of the present invention, illustrated in FIG. 4, insert 16 may include protrusions 18 in the form of a knob or a plurality of knobs or fingers 18, extending in a direction substantially transverse to planar portion 20 of insert 16. Knobs or fingers 18 are also at least semi-rigid, for example rigid, and may be formed, for example, from metal, wood, synthetic rubber, natural rubber or a polymeric material. According to this embodiment of the healthcare device 10 of the present invention, knobs or fingers 18 may be arranged to contact a plurality of predefined pressure or acupressure points of a user.

According to an additional embodiment of the present invention, protrusion 18 extending from insert 16 may be in the form of a neck frame 18, illustrated in FIG. 5. Neck frame 18 is configured for comfortably supporting the head and neck of a user. Neck frame 18 is adjustable in a horizontal and/or vertical direction in order to accommodate users having necks of different widths and/or lengths.

Referring now to FIG. 6, there is shown an embodiment of the present invention having a plurality of recesses 14 which are each sized and shaped to receive at least one insert 16. Accordingly, it is feasible to have, for example two inserts 16, each having protrusions 18 which are sized and/or shaped differently.

The present invention further provides a method of using a healthcare device 10. According to the method of the present invention, base member 12, including one or more recesses 14 is mounted to a wall. One or more inserts 16 having a shape and size corresponding to the recesses 14 is positioned within or inserted into recesses 14. A body surface is selectively positioned against and pressure selectively applied to one or more protrusions 18 extending from inserts 16.

Optionally, the method according to the present invention further includes the step of securely fastening inserts 16 within recesses 14 using any of a number of known fasteners. Alternatively, the inserts may be firmly secured within the recesses using a snap fit or compression fit.

Further, the protrusion is in the form of a neck frame, the method of the present invention further includes the step of adjusting the neck frame to fit firmly around the neck of a user in a horizontal and/or vertical direction.

While this invention has been described with respect to at least one embodiment, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains and which fall within the limits of the appended claims.

What is claimed is:

1. A personal healthcare device, comprising:
a base member mountable to a wall, said base member including at least one recess; and
at least one insert, each said insert being positioned within a corresponding said recess and having a size and shape of said corresponding recess, each said insert including at least one protrusion extending from said insert in a direction away from said base member, each said protrusion being configured for selectively making contact with and applying pressure to a surface of a user.

2. The personal healthcare device according to claim 1, wherein said at least one insert includes a substantially planar portion.

3. The personal healthcare device according to claim 2, wherein said at least one protrusion extending from said insert is a neck frame configured for supporting the head and neck of a user.

4. The personal healthcare device according to claim 3, wherein said neck frame is adjustable from side to side.

5. The personal healthcare device according to claim 4, wherein said at least one insert is securely coupled with said base member with a fastener.

6. The personal healthcare device according to claim 2, wherein said at least one protrusion extending from said insert is a plurality of protrusions.

7. The personal healthcare device according to claim 6, wherein said plurality of protrusions are a plurality of semi-rigid bristles for exfoliating skin.

8. The personal healthcare device according to claim 7, wherein said semi-rigid bristles are one of boar bristles, horse hair bristles, nylon bristles and stainless steel bristles.

9. The personal healthcare device according to claim 6, wherein said plurality of protrusions are arranged to contact a plurality of predefined pressure points of the user.

10. The personal healthcare device according to claim 6, wherein said plurality of protrusions are a plurality of knobs extending in a direction substantially transverse to said planar portion of said at least one insert.

11. The personal healthcare device according to claim 6, wherein said plurality of protrusions are formed from one of synthetic rubber, natural rubber, wood and polymeric material.

12. The personal healthcare device according to claim 1, wherein said at least one insert is a plurality of inserts and said at least one recess is a plurality of recesses, each of said plurality of inserts having a size and shape corresponding to at least one of said plurality of recesses.

13. The personal healthcare device according to claim 12, wherein said plurality of inserts each include at least one protrusion extending away from said base member.

14. A method of using a personal healthcare device, the method comprising the steps of:
mounting a base member to a wall, said base member including at least one recess; and
inserting at least one insert into said at least one recess, said at least one insert having a size and shape corresponding with a size and shape of said at least one recess, said at least one insert including at least one protrusion extending from said insert in a direction away from said base member, each said protrusion being configured for selectively making contact with and applying pressure to a surface of a user;
securing said at least one insert within said at least one recess;
selectively positioning a body surface against said at least one protrusion;
selectively applying pressure against said at least one protrusion.

15. The method according to claim 14, wherein said base member includes at least one second said recess, said method further comprising the step of inserting a second said insert into said at least one second recess, said second insert having a shape and size corresponding to said at least one second recess.

16. The method according to claim 15, wherein said second insert includes at least one said protrusion which is different than another at least one said protrusion of another said insert.

17. The method according to claim 14, said step of securing said at least one insert includes using at least one fastener to secure said at least insert within said at least one recess.

18. The method according to claim 14, wherein said at least one protrusion is an adjustable neck frame, said method further comprising the step of adjusting said neck frame to fit securely around the neck of a user.

19. The method according to claim 14, said securing step further comprising using a snap fit to secure said at least one insert within said at least one recess.

20. The method according to claim 14, further comprising the step of securing said at least one insert within said at least one recess with a pressure fit.
